(19) United States
(12) Patent Application Publication
(10) Pub. No.: US 2026/0042767 A1
(43) Pub. Date: Feb. 12, 2026

(54) TRICYCLIC COMPOUNDS FOR TREATMENT OF HSV

(71) Applicant: Assembly Biosciences, Inc., South San Francisco, CA (US)

(72) Inventors: Hassan PAJOUHESH, South San Francisco, CA (US); Michael WALKER, South San Francisco, CA (US); Min ZHONG, South San Francisco, CA (US); Jian ZHANG, South San Francisco, CA (US); Mark BURES, South San Francisco, CA (US); Thomas P. STRATTON, Foster City, CA (US)

(21) Appl. No.: 19/292,855

(22) Filed: Aug. 6, 2025

Related U.S. Application Data
(60) Provisional application No. 63/680,428, filed on Aug. 7, 2024.

(51) Int. Cl. C07D 487/04; A61K 31/519; A61P 31/22

(52) U.S. Cl. CPC C07D 487/04; A61K 31/519; A61P 31/22

(57) ABSTRACT

The present disclosure provides, in part, tricyclic compounds, and pharmaceutical compositions thereof, and methods of the treatment and prophylaxis of HSV infections.

### CROSS REFERENCE TO RELATED APPLICATION

This application claims the benefit of U.S. Provisional Application No. 63/680,428, filed Aug. 7, 2024, which is incorporated herein by reference in its entirety.

### BACKGROUND

Human herpes viruses are large-enveloped double-stranded DNA viruses that share the characteristic of establishing life-long infections in humans. This is accomplished by their ability to exist in the host either as a symptom free latent infection, where the virus lies dormant or, following activation, as a lytic infection with associated symptoms. These viral infections have widespread, worldwide prevalence and it is notable that over 90% of all humans are chronically infected with more than one human herpes virus.

Human herpes viruses are classified into three subfamilies (i.e., a, R and y) based upon their biological characteristics and the family consists of eight members, i.e., Herpes Simplex Virus subtype type 1 and 2 (HSV1, HSV2), Varicella Zoster Virus (VZV), Epstein-Barr virus (EBV), Cytomegalovirus (CMV), and human herpes viruses 6-8 (HHV 6-8).

HSV1 and 2 infections can cause disease in immune competent individuals. Both subtypes cause cutaneous genital/anal and orolabial/nasal cavity (cold sore) lesions, although HSV2 is more commonly associated with the former and HSV1 the latter such that >80% of genital infections are believed to be caused by HSV2. Globally, over 500 million people have genital herpes infections. Symptoms vary but are typically most severe on first time of infection and can last for weeks to months. Approximately 50 to 80% of the world's population have orolabial HSV infection, which is the main cause of cold sores. HSV, and particularly HSV1, can also cause lesions on the fingers (Whitlows) and other areas of the skin.

The vast majority of HSV infected individuals will not experience any noticeable symptoms. However, some will experience recurrent outbreaks of infection. In the USA, 20 to 40% of the population will get recurrent labial HSV lesions. Significantly, orolabial cold sores and Whitlow's provide a very easy route for transmission of the virus to other individuals which can lead to rarer but much more serious HSV-related pathologies. For example, HSV-related ocular keratitis is a major cause of blindness. HSV can also cause encephalitis in neonates which is a life-threatening condition. Other disorders also believed to be caused by HSV include herpes gladiatorum, Mollaret's meningitis and possibly Bell's palsy.

Primary infection with, or reactivation of an existing herpes virus infection, can be a major cause of disease in immunocompromised individuals. Key at-risk immunocompromised populations include patients undergoing solid organ or stem cell transplantation, individuals with HIV/AIDS, and ICU patients.

Presently, there is no cure for HSV. Medicines have been developed that can to some degree prevent or shorten outbreaks, but there is a need for improved therapies for treating HSV infection and inhibiting viral replication.

Currently, nucleoside analogues, such as acyclovir and its prodrugs, e.g., valacyclovir and famciclovir, are used as agents against herpes viruses such as HSV. In order to exert their effects, these nucleoside analogues must first be phosphorylated by viral thymidine kinase (TK) and then subsequently converted by cellular kinases to the nucleoside triphosphate, which inhibits the activity of the viral DNA polymerase. If the virus has no functionally active TK, as is the case, for example, with resistant HHV1 mutants or with TK-negative viruses, the active substance is unable to exert its effects.

Nucleoside analogues are clinically administered at a dose as high as several hundred in mg to several grams per day and even in high doses, and over long treatment durations, these compounds do not completely prevent recurrent outbreaks of symptoms from HSV infection. High doses also lead to increased levels of adverse effects.

Viral shedding is also common in HSV patients and can asymptomatically facilitate the transmission of HSV to more individuals. Nucleoside analogues do little to address this and long-term suppressive treatment, e.g., with valacyclovir has been shown to reduce transmission risk only by 46%. Since the nucleoside analogues can incorporate into the genome DNA of a host via the host DNA polymerase, the mutagenicity of these agents is also a concern, as documented for the nucleoside analogue, ganciclovir.

Given the inadequacy of existing treatments, there is an urgent medical need to develop improved, well-tolerated anti-herpes treatments.

A class of compounds being investigated for HSV treatment are the helicase-primase inhibitors. Helicase-primase inhibitors are antiviral agents with a novel mechanism of action against HSV1 and 2. They inhibit the viral heterotrimeric complex consisting of helicase, primase, and cofactor subunits that have functions essential for viral DNA replication. They are not nucleoside analogues and do not require phosphorylation by TK to inhibit HSV replication and they are therefore potentially active against TK-deficient HSV, which as described above, is a major mechanism of resistance to nucleoside analogues, such as acyclovir.

Two examples of helicase-primase inhibitors are BILS-179 BS and amenamevir (Katsumata et al. (2018) Biochem Pharm 158 p 201-206). BILS-179 BS has been dosed orally but was suspended from early clinical trials due to adverse events. Another example is pritelivir, a thiazolylamide derivative with the chemical name N-[5-(aminosulfonyl)-4-methyl-1,3-thiazol-2-yl]-N-methyl-2-[4-(2-pyridinyl)-phenyl]acetamide.

WO2000053591 discloses thiazolyl urea derivatives of formula (I), to a method for the production thereof, and to their use as medicaments, in particular, as antiviral medicaments.

WO2000076966 discloses indolinylanide derivatives, a method for the production thereof, and the use of said compounds as medicaments, particularly as antiviral drugs.

WO2001047904 discloses thiazoll arnide derivatives, to a method for producing them and to their use as medicaments, especially as antiviral medicaments.

WO2017174640 discloses aminothiazole derivatives, to a process for their preparation and to their use as medicaments, in particular as antiviral medicaments.

WO2019068817 discloses substituted thiazole antiviral compounds with specific stereo-configuration, especially to specific novel enantiomers, to a process for their preparation and to their use as medicaments, in particular as antiviral medicaments.

WO2021126804 relates to indazole derivatives and compositions comprising at least one indazole derivative, and methods of using the indazole derivatives for treating or preventing a herpesvirus infection in a patient.

WO2023225162 relates to indolinyl compounds that inhibit viral helicase-primase; the use of the compounds for the preparation a medicament for the treatment of diseases and/or condition through inhibiting viral helicase-primase; use of those compounds in the treatment of viral infections; and intermediates for its preparation and to pharmaceutical compositions containing those compounds.

WO2024049760 discloses, in part, cyclic urea thiazolyl compounds, pharmaceutical compositions thereof, and methods of the treatment and prophylaxis of HSV infections.

There is still a need for additional antiviral compounds for the treatment and prophylaxis of HSV infections that have an improved profile with respect to safety, potency, selectivity and/or bioavailability.

### SUMMARY OF THE INVENTION

In one embodiment, the present disclosure provides a compound of Formula I

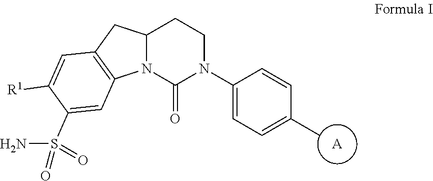

or a pharmaceutically acceptable salt thereof, wherein the variables are as described herein.

In another aspect, the disclosure provides pharmaceutical compositions comprising a compound of Formula I, or a pharmaceutically acceptable salt thereof, and a pharmaceutically acceptable excipient.

In another aspect, the disclosure provides a method of treating an HSV infection in a subject in need thereof, comprising administering to the subject a therapeutically effective amount of compound of Formula I, or a pharmaceutically acceptable salt thereof.

In another aspect, the disclosure provides a method of treating an HSV infection in a subject in need thereof, comprising administering to the subject a pharmaceutical composition comprising a therapeutically effective amount of a compound of Formula I, or a pharmaceutically acceptable salt thereof, and a pharmaceutically acceptable excipient.

### DETAILED DESCRIPTION OF THE INVENTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The term “alkyl” as used herein refers to a saturated straight or branched hydrocarbon. Exemplary alkyl groups include, but are not limited to, straight or branched hydrocarbons of 1-6 or 1-4 carbon atoms, referred to herein as C₁₋₆ alkyl and C₁₋₄ alkyl, respectively. Examples include, but are not limited to, methyl, ethyl, n-propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, t-butyl, n-pentyl, isopentyl, neopentyl, and n-hexyl.

The terms “halo” or “halogen” as used herein refer to F, Cl, Br or I.

The term “haloalkyl” as used herein refers to an alkyl group substituted with one or more halogen atoms. Exemplary haloalkyl groups include, but are not limited to, a C₁₋₆alkyl or C₁₋₄alkyl substituted with one or more halo groups, referred to herein as haloC₁₋₆alkyl and haloC₁₋₄alkyl, respectively. For example, haloC₁₋₆alkyl refers to a straight or branched alkyl group of 1-6 carbon atoms substituted with one or more halogen atoms. Examples include, but are not limited to, —CH₂F, —CHCl₂, —CHF₂, —CF₃, CF₃CH₂—, CH₃CF₂—, CF₃CCl₂— and CF₃CF₂—.

The terms “Individual,” “patient,” or “subject” are used interchangeably and include any animal, including mammals, preferably mice, rats, other rodents, rabbits, dogs, cats, swine, cattle, sheep, horses, or primates, and most preferably humans. The compounds or pharmaceutical compositions of the disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, dogs, primates, and the like). The mammal treated in the methods of the disclosure is desirably a mammal in which treatment of HSV infection is desired.

The term “modulation” includes antagonism (e.g., inhibition), agonism, partial antagonism and/or partial agonism.

The term “Pharmaceutically acceptable” includes molecular entities and compositions that do not produce an adverse, allergic, or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, fillers, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable excipients.

The term “pharmaceutically acceptable salt(s)” as used herein refers to salts of acidic or basic groups that may be present in compounds used in the compositions. Compounds included in the present compositions that are basic in nature can form a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The term “therapeutically effective amount” or “effective amount” as used herein refers to the amount of the subject compound that will elicit the biological or medical response of a tissue, system or animal, (e.g., mammal or human) that is being sought by the researcher, veterinarian, medical doctor or other clinician. The compounds or pharmaceutical compositions of the disclosure are administered in therapeutically effective amounts to treat a disease. Alternatively, a therapeutically effective amount of a compound is the quantity required to achieve a desired therapeutic and/or prophylactic effect.

The term “treating” includes any effect, e.g., lessening, reducing, modulating, or eliminating, a viral infection, that results in the improvement of the disease.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic ring may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantiomeric and diastereoselective transformations and may involve the use of chiral auxiliaries. For examples, see Carreira and Kvaerno, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like, and it is intended that the disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine and chlorine, such as ²H, ³H₂, ¹³C, ¹⁴C, ¹⁵N, ¹⁸O, ¹⁷O ³¹P, ³²p, ³⁵S, ¹⁸F, and ³⁶Cl, respectively. For example, a compound of the disclosure may have one or more H atom replaced with deuterium.

Certain isotopically labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure can generally be prepared by following procedures analogous to those disclosed in the examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al., Nature Reviews Drug Discovery 2008, 7, 255).

### Tricyclic Compounds of the Present Invention

In one aspect, the present disclosure provides a compound of Formula I

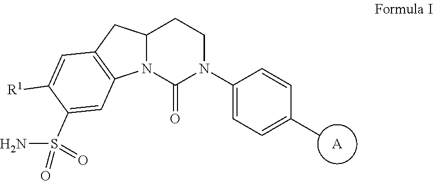

or a pharmaceutically acceptable salt thereof, wherein:

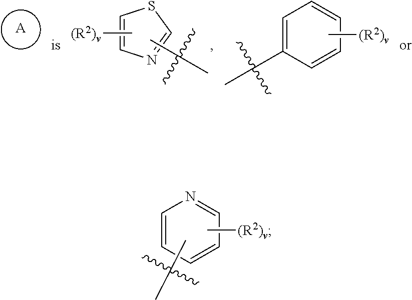

R¹ is hydrogen, CN, halo or C₁₋₄alkyl;
R² is independently selected for each occurrence from the group consisting of halo, CN, C₁₋₄alkyl and haloC₁₋₄alkyl; and
v is 0, 1 or 2;

The following embodiments further describe a compound of Formula I, or a pharmaceutically acceptable salt thereof. It will be appreciated that all chemically allowable combinations of the embodiments described herein are envisioned as further embodiments of the invention.

In certain embodiments, is

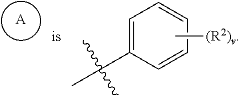

In certain embodiments,

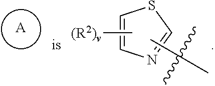

In certain embodiments,

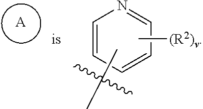

In certain embodiments,

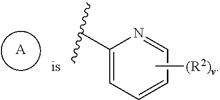

In certain embodiments, R¹ is F.

In certain embodiments, v is 1 or 2 and R² is F for each occurrence.

### Methods of Use

The compounds according to the present invention are useful for the treatment and prophylaxis of disorders caused by herpes viruses, in particular Herpes simplex viruses.

In one aspect, the present invention provides a method for the treatment or prophylaxis of an HSV infection in a subject in need thereof, the method comprising: administering to the subject a therapeutically effective amount of a compound of the invention, or a pharmaceutically acceptable salt thereof.

In some embodiment, the infection is a Herpes simplex infection.

In some embodiment, the infection is an HSV-1 infection.

In some embodiment, the infection is an HSV-2 infection.

In some embodiments, the infection is a Herpes simplex infection and the subject displays symptoms such as Herpes labialis, Herpes genitalis, HSV-related keratitis, encephalitis, or pneumonia.

In another embodiment, the infection is a Herpes simplex infection and the subject displays symptoms such as suppressed immune system (for example AIDS patients, cancer patients, patients having a genetic immunodeficiency, transplant patients).

In another embodiment, the infection is a Herpes simplex infection, and the subject is a new-born child or infant.

In another aspect, the present invention provides a method for suppressing recurrence of HSV symptoms or outbreaks in a subject in need thereof, the method comprising: administering to the subject a therapeutically effective amount of a compound of the invention, or a pharmaceutically acceptable salt thereof.

In some embodiment, the infection is a Herpes simplex infection.

In some embodiment, the infection is an HSV-1 infection.

In some embodiment, the infection is an HSV-2 infection.

In some embodiment, the subject is a herpes-positive patient.

In some embodiment, the subject is a herpes-simplex-positive patient.

In another aspect, the present invention provides a method for the treatment or prophylaxis of an HSV infection in a subject in need thereof, the method comprising: administering to the subject a therapeutically effective amount of a compound of the invention, or a pharmaceutically acceptable salt thereof, wherein: the infection is resistant to nucleosidic antiviral therapy.

In one embodiment, the infection is a Herpes simplex infection.

In some embodiment, the infection is a Herpes simplex infection.

In another embodiment, the subject is a herpes-positive patient.

In another embodiment, the nucleosidic antiviral therapy is selected from the group consisting of acyclovir, penciclovir, famciclovir, ganciclovir and valacyclovir.

In another aspect, the present invention provides a compound for the use as a medicament.

### Combination Therapies

The compounds according to the present invention are also useful for the treatment and prophylaxis of disorders caused by herpes viruses, in particular Herpes simplex viruses, in combination with other active ingredients.

In one aspect, the present invention provides a method for the treatment or prophylaxis of an HSV infection in a subject in need thereof, the method comprising: administering to the subject a therapeutically effective amount of a compound of the invention, or a pharmaceutically acceptable salt thereof, in combination with an antiviral agent.

In some embodiments, the antiviral agent is selected from the group consisting of acyclovir, penciclovir, famciclovir, ganciclovir and valacyclovir, foscarnet and trifluridine.

In some embodiment, the infection is a Herpes simplex infection.

In some embodiment, the infection is an HSV-1 infection.

In some embodiment, the infection is an HSV-2 infection.

In some embodiments, the infection is a Herpes simplex infection and the subject displays symptoms such as Herpes labialis, Herpes genitalis, HSV-related keratitis, encephalitis, or pneumonia.

In another embodiment, the infection is a Herpes simplex infection and the subject displays symptoms such as suppressed immune system (for example AIDS patients, cancer patients, patients having a genetic immunodeficiency, transplant patients).

In another embodiment, the infection is a Herpes simplex infection, and the subject is a new-born child or infant.

In another aspect, the present invention provides a method for suppressing recurrence of HSV symptoms or outbreaks in a subject in need thereof, the method comprising: administering to the subject a therapeutically effective amount of a compound of the invention, or a pharmaceutically acceptable salt thereof, in combination with an antiviral agent.

In some embodiments, the antiviral agent is selected from the group consisting of acyclovir, penciclovir, famciclovir, ganciclovir and valacyclovir, foscarnet and trifluridine.

In some embodiment, the infection is a Herpes simplex infection.

In some embodiment, the infection is an HSV-1 infection.

In some embodiment, the infection is an HSV-2 infection.

In some embodiment, the subject is a herpes-positive patient.

In some embodiment, the subject is a herpes-simplex-positive patient.

In another aspect, the present invention provides a compound for the use as a medicament.

In another aspect, the present invention provides a method for the treatment or prophylaxis of an HSV infection in a subject in need thereof, the method comprising: administering to the subject a therapeutically effective amount of a compound of the invention, or a pharmaceutically acceptable salt thereof, in combination with a corticosteroid.

In some embodiment, the infection is a Herpes simplex infection.

In some embodiment, the infection is an HSV-1 infection.

In some embodiment, the infection is an HSV-2 infection.

In some embodiments, the infection is a Herpes simplex infection and the subject displays symptoms such as Herpes labialis, Herpes genitalis, HSV-related keratitis, encephalitis, or pneumonia.

In another embodiment, the infection is a Herpes simplex infection and the subject displays symptoms such as suppressed immune system (for example AIDS patients, cancer patients, patients having a genetic immunodeficiency, transplant patients).

In another embodiment, the infection is a Herpes simplex infection, and the subject is a new-born child or infant.

In another aspect, the present invention provides a method for suppressing recurrence of HSV symptoms or outbreaks in a subject in need thereof, the method comprising: administering to the subject a therapeutically effective amount of a compound of the invention, or a pharmaceutically acceptable salt thereof, in combination with a corticosteroid.

In some embodiment, the infection is a Herpes simplex infection.

In some embodiment, the infection is an HSV-1 infection.

In some embodiment, the infection is an HSV-2 infection.

In some embodiment, the subject is a herpes-positive patient.

In some embodiment, the subject is a herpes-simplex-positive patient.

In another aspect, the present invention provides a compound for the use as a medicament.

### Formulations and Administration

The compounds on the invention can be converted in a known manner into the customary formulations, such as tablets, sugar-coated tablets, pills, granules, aerosols, syrups, emulsions, suspensions, and solutions, using inert, nontoxic, pharmaceutically suitable carriers and solvents. Here, the therapeutically active compound should in each case be present in a concentration of about 0.5 to 90% by weight of the total mixture, i.e., in amounts which are sufficient to achieve the dosage range indicated.

The formulations are prepared, for example, by extending the active compounds with solvents and/or excipients, if appropriate using emulsifiers and/or dispersants, it being possible, for example, if the diluent used is water, to use, if appropriate, organic solvents as auxiliary solvents.

Administration is carried out in a customary manner, including orally, parenterally, topically, perlingually or intravenously.

In the case of parenteral administration, solutions or suspensions of the active compounds using suitable liquid carrier and excipients can be employed.

In general, it has proved advantageous in the case of intravenous administration to administer amounts of from approximately 0.001 to 20 mg/kg, preferably approximately 0.01 to 10 mg/kg, of bodyweight to achieve effective results, and in the case of oral administration the dose is approximately 0.01 to 30 mg/kg, preferably 0.1 to 20 mg/kg, of bodyweight.

In some instances, it may be necessary to depart from the amounts mentioned, namely depending on the bodyweight or on the type of administration route, on the individual response to the medicament, the manner of its formulation and the time or interval at which administration takes place. Thus, in some cases it may be adequate to manage with less than the abovementioned minimum amount, while in other cases the upper limit mentioned must be exceeded. In the case of the administration of relatively large amounts, it may be advisable to divide this into several individual administrations over the course of the day.

If appropriate, it may be useful to combine the compounds according to the invention with other active substances, in particular antiviral active substances.

The compounds used in the present invention can be in the form of a pharmaceutically acceptable salt, cocrystal or a solvate. The term “pharmaceutically acceptable salts” refers to salts prepared from pharmaceutically acceptable non-toxic bases or acids, including inorganic bases or acids and organic bases or acids. In case the compounds of the present invention contain one or more acidic or basic groups, the invention also comprises their corresponding pharmaceutically or toxicologically acceptable salts, in particular their pharmaceutically utilizable salts. Thus, the compounds of the present invention which contain acidic groups can be used according to the invention, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The compounds of the present invention which contain one or more basic groups, i.e., groups which can be protonated, can be used according to the invention in the form of their addition salts with inorganic or organic acids. Examples of suitable acids include hydrogen chloride, hydrogen bromide, phosphoric acid, sulfuric acid, nitric acid, methanesulfonic acid, p-toluenesuifonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present invention simultaneously contain acidic and basic groups in the molecule, the invention also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

Depending on the substitution pattern, the compounds according to the invention can exist in stereoisomeric forms which either behave as image and mirror image (enantiomers), or which do not behave as image and mirror image (diastereomers). The invention relates both to the enantiomers or diastereomers and their respective mixtures. Like the diastereomers, the racemic forms can be separated into the stereoisomerically uniform components in a known manner.

The scope of the invention includes those compounds which are only converted into the actual active compounds of the Formulas I and once inside the body (so-called prodrugs).

In practical use, the compounds used in the present invention can be combined as the active ingredient in intimate admixture with a pharmaceutical carrier according to conventional pharmaceutical compounding techniques. The carrier may take a wide variety of forms depending on the form of preparation desired for administration, e.g., oral, or parenteral {including intravenous). In preparing the compositions for oral dosage form, any of the usual pharmaceutical media may be employed, such as, for example, water, glycols, oils, alcohols, flavouring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray or as eye drops.

The tablets, pills, capsules, and the like may also contain a binder such as hydroxypropyl methylcellulose, or polyvinylpyrrolidone; diluent or fillers such as microcrystalline cellulose, dicalcium phosphate, lactose, or mannitol; a disintegrating agent such as croscarmellose sodium, polyvinylpyrrolidone, or sodium starch glycolate; a lubricant such as magnesium stearate or sodium stearyl fumarate; a glidant such as silicon dioxide; and a sweetening agent such as sucrose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

The compounds used in the present invention may also be administered parenterally. Solutions or suspensions of these active compounds can be prepared in water suitably mixed with a surfactant such as hydroxypropyl cellulose, sodium lauryl sulfate, or polysorbate. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral (including intravenous), ocular, pulmonary, nasal, and the like may be employed. Dosage forms include tablets, troches, dispersions, suspensions, solutions, capsules, creams, ointments, aerosols, and the like. Compounds of the present invention can be administered orally or as eye drop. The compounds of the present invention can also be administered orally. The effective dosage of active ingredient employed may vary depending on the particular compound employed, the mode of administration, the condition being treated, and the severity of the condition being treated. Such dosage may be ascertained readily by a person skilled in the art.

The compounds of the present invention can also be present in combination with additional active ingredients, in particular, with one or more active ingredients exhibiting advantageous effects in the treatment of any of the disorders or diseases as described herein. The compounds of the present invention can be present in a composition in combination with at least one further active substance being effective in treating a disease or disorder associated with viral infections (antiviral active compounds), preferably a disease or disorder being associated with viral infections caused by herpes viruses, such as in particular by Herpes simplex viruses (i.e., combination therapy). The at least one further active substance being effective in treating a disease or disorder associated with viral infections (antiviral active compounds) are preferably selected from the group consisting of nucleosidic drugs such as acyclovir, valacyclovir, penciclovir, ganciclovir, famciclovir and trifluridine, as well as compounds such as foscarnet and cidofovir.

Accordingly, the present invention further relates to a pharmaceutical composition comprising one or more of the compounds as described herein and at least one pharmaceutically acceptable carrier and/or excipient and/or at least one further active substance being effective in treating a disease or disorder associated with viral infections (antiviral active compounds).

The novel active compounds can be converted in a known manner into customary formulations, such as tablets, caplets, sugar-coated tablets, pills, granules, aerosols, syrups, pharmaceutically suitable carriers, and solvents. Here, the therapeutically active compound should in each case be present in a concentration of about 0.1 to 90% by weight of the total mixture, i.e., in amounts which are sufficient to achieve the dosage range indicated.

The formulations are prepared, for example, by extending the active compounds with solvents and/or excipients, if appropriate using emulsifiers and/or dispersants, if being possible, for example, if the diluent used is water, to use, if appropriate, organic solvents as auxiliary solvents.

Administration is carried out in a customary manner, preferably orally, parenterally or topically, in particular perlingually or intravenously.

In the case of parenteral administration, solutions or suspensions of the active compounds using suitable liquid carrier materials can be employed.

In general, it has proved advantageous in the case or intravenous administration to administer amounts of from approx. 0.001 to 20 mg/kg, preferably approx. 0.01 to 10 mg/kg of bodyweight to achieve effective results, and in the case of oral administration the dose is approx. 0.01 to 30 mg/kg, preferably 0.1 to 20 mg/kg of body weight.

In spite of this, it may be necessary, if appropriate, to depart from the amounts mentioned, namely depending on the bodyweight or on the type of the administration route, on the individual response to the medicament, the manner of its formulation and the time or interval at which administration takes place. Thus, in some cases it may be adequate to manage with less than the abovementioned minimum amount, while in other cases the upper limit mentioned must be exceeded. In the case of administration of relatively large amounts it may be advisable to divide this into several individual administrations over the course of the day.

### Examples

The compounds described herein can be prepared in several ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

At least some of the compounds identified as “intermediates” herein are contemplated as compounds of the disclosure.

### Abbreviations

AcOH Acetic acid
ACN Acetonitrile
Boc₂O Di-tert-butyl dicarbonate
nBuLi n-Butyllithium
DCM Dichloromethane
DIAD Diisopropyl azodicarboxylate
DIEA Diisopropyl ethylamine
DMF N, N-Dimethylformamide
DMSO Dimethyl sulfoxide
DPPF 1,1′-Bis(diphenylphosphino)ferrocene
EtOAc Ethyl acetate
Et₃N Triethylamine
HATU Hexafluorophosphate Azabenzotriazole Tetramethyl Uronium
h, hr Hour(s)
HPLC High performance liquid chromatography
LCMS Liquid chromatography-mass spectrometry
MeOH Methanol
NMON Methyl morpholine-N-Oxide
NBS N-Bromosuccinimide
PE Petroleum ether
iPrOH Isopropanol
rt, r.t. Room temperature
SFC Supercritical Fluid Chromatography
TEA Triethylamine
TBAI Tetrabutylammonium iodide
TBAB Tetrabutylammonium bromide
TFA Trifluoroacetic acid
THF Tetrahydrofuran
TLC Thin-layer chromatography
XPhos 2-Dicyclohexylphosphino-2′,4′,6′-triisopropylbiphenyl
   Following LCMS Method have been Used for the Analysis of Final Compounds

Method A: X-Bridge BEH C-18 (3×50 mm×2.5 mm); Mobile phase: A; 0.025% formic acid in H₂O; B; CH₃CN; Injection volume:2 μL; Flow rate:1.2 mL/min, column temperature: 50° C.; Gradient program: 2% B to 98% B in 2.2 min, hold till 3 min, at 3.2 min B conc. is 2% till up to 4 min.

Method B: X-select CSH 18 (3×50 mm×2.5 mm); Mobile phase: A; 0.025% formic acid in H₂O; B; CH₃CN; Injection volume:2 μL; Flow rate:1.2 mL/min, column temperature: 50° C.; Gradient program: 0% B to 98% B in 2 min, hold till 3 min, at 3.2 min B conc. is 0% till up to 4 min.

Method C: X-select CSH 18 (3×50 mm×2.5 mm); Mobile phase: A; 0.05% formic acid in H₂O:CH₃CN (95:5); B; 0.05% formic acid in CH₃CN; Injection volume: 2 μL; Flow rate: 1.2 mL/min, column temperature: 50° C.; Gradient program: 0% B to 98% B in 2 min, hold till 3 min, at 3.2 min B conc. is 0% till up to 4 min.

Method D: X-select CSH C₁₈ (3×50 mm×2.5 mm); Mobile phase: A; 2 mM in Ammonium Bicarbonate; B; CH₃CN; Injection volume:2 μL; Flow rate:1.2 mL/min, column temperature: 50° C.; Gradient program: 0% B to 98% B in 2 min, hold till 3 min, at 3.2 min B conc. is 0% till up to 4 min.

Method E: X-select CSH 18 (3×50 mm×2.5 mm); Mobile phase: A; 0.05% formic acid in H₂O; B; CH₃CN; Injection volume: 2 μL; Flow rate:1.5 mL/min, column temperature: 50° C.; Gradient program: 0% B to 100% B in 1.5 min, hold till 2.2 min, at 2.6 min B conc. is 0% till up to 3 min.

### Example 1. 7-Fluoro-1-oxo-2-(4-(pyridin-2-yl)phenyl)-1,2,3,4,4a,5-hexahydropyrimido[1,6-a]indole-8-sulfonamide

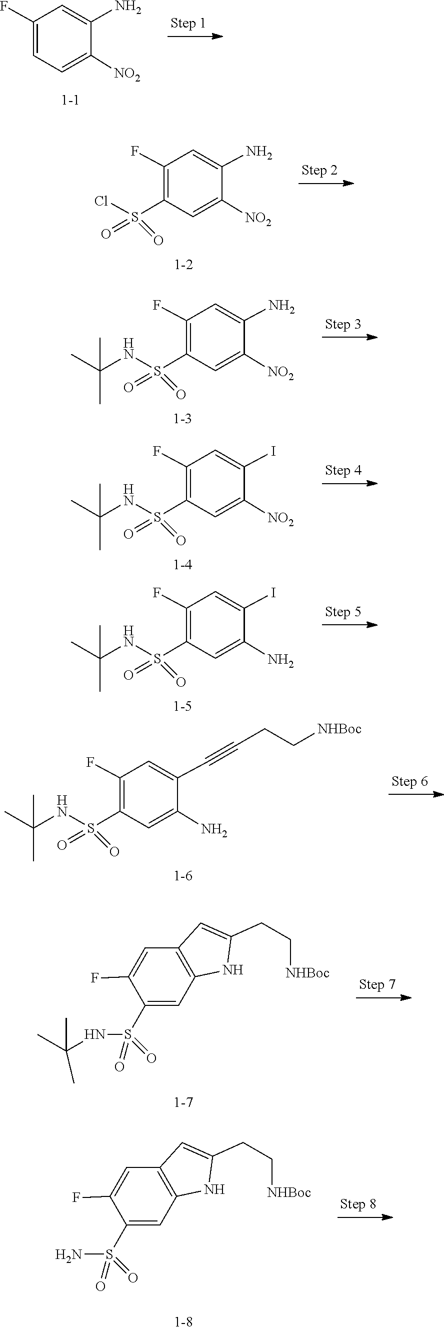

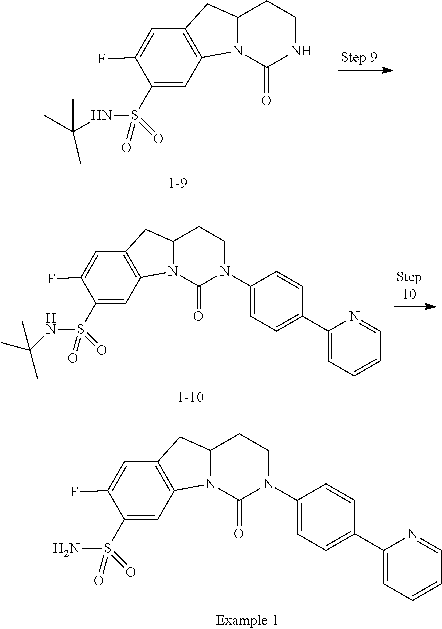 [SMILES: CC(C)(C)NS(=O)(=O)c1cc2c(cc1F)CC1CCNC(=O)N21 ; CC(C)(C)NS(=O)(=O)c1cc2c(cc1F)CC1CCN(c3ccc(-c4ccccn4)cc3)C(=O)N21 ; NS(=O)(=O)c1cc2c(cc1F)CC1CCN(c3ccc(-c4ccccn4)cc3)C(=O)N21]

Step 1. Synthesis of 4-amino-2-fluoro-5-nitrobenzenesulfonyl chloride (1-2). To a stirred solution of compound 1-1 (20 g, 128.11 mmol) was added chlorosulfonic acid (100 mL) at 0° C. The reaction mixture stirred at 120° C. for 6 h. Upon completion (monitored by TLC), the mixture was cooled to room temperature, diluted with ice-cold water, and the resulting solid was filtered. The filtrate was concentrated and the residue was dried in vacuo to give compound 1-2 (20 g) as a brown solid, which was used in the next step without further purification. TLC: 40% EtOAc/heptane (R_{f}: 0.3). MS (ESI): calcd. for C₆H₄ClFN₂O₄S: 253.96; Found: 253.17 [M−1]⁻.

Step 2. Synthesis of 4-amino-N-(tert-butyl)-2-fluoro-5-nitrobenzenesulfonamide (1-3). To a stirred solution of compound 1-2 (20 g, 78.55 mmol) in THF (200 mL) at 0° C. under nitrogen atmosphere, was added DIPEA (31.1 g, 235.65 mmol) followed by 2-methylpropan-2-amine (11.5 g, 157.10 mmol). The resulting reaction mixture was slowly warmed to room temperature and allowed to stir at 70° C. for 6 h. After completion (monitored by TLC), the reaction mixture was concentrated under reduced pressure. The crude product was triturated with diethyl ether and dried under reduced pressure to give compound 1-3 (18 g, crude) as a yellow solid, which was used in the next step without further purification. TLC: 40% EtOAc/heptane (R_{f}: 0.3). MS (ESI): calcd. for C₁₀H₁₄FN₃O₄S: 291.07; Found: 290.32 [M−1]⁻. ¹H NMR (401 MHz, DMSO-d6): δ 8.39 (d, J=7.8 Hz, 1H), 8.09 (s, 2H), 7.72 (s, 1H), 6.87 (d, J=12.2 Hz, 1H), 1.14 (s, 9H) ppm.

Step 3. Synthesis of N-(tert-butyl)-2-fluoro-4-iodo-5-nitrobenzenesulfonamide (1-4). To a stirred solution of compound 1-3 (18 g, 61.79 mmol) in ACN (200 mL) at 0° C. was added CuI (23.54 g, 123.58 mmol) and resulting mixture stirred for 10 min at the same temperature. Tert-butyl nitrite (14.16 g, 123.58 mmol) was then added dropwise. The reaction mixture was slowly warmed to room temperature and stirred at 70° C. for 5 h. After completion (monitored by TLC), the mixture was filtered through celite, diluted with water, and washed with EtOAc. The combined organic layers were treated with 1N HCl, extracted with EtOAc, dried over anhydrous Na₂SO₄, and concentrated under reduced pressure. The crude product was purified by CombiFlash column chromatography (eluting with 10-30% EtOAc in heptane) to give compound 1-4 (8 g, 32.19%) as a yellow solid. TLC: 20% EtOAc/heptane (R_{f}: 0.5). MS (ESI): calcd. for C₁₀H₁₂FIN₂O₄S: 401.95; Found: 401.23 [M−1]⁻. ¹H NMR (400 MHz, CDCl₃): δ 8.45 (d, J=6.6 Hz, 1H), 7.91 (d, J=8.3 Hz, 1H), 4.78 (br s, 1H), 1.28 (s, 9H) ppm.

Step 4. Synthesis of 5-amino-N-(tert-butyl)-2-fluoro-4-iodobenzenesulfonamide (1-5). To a stirred solution of compound 1-4 (8 g, 19.89 mmol) in EtOH: H₂O (80:20 mL) was added NH₄Cl (5.3 g, 99.46 mmol) followed by Iron (5.56 g, 99.46 mmol) at room temperature. The reaction mixture was stirred at 70° C. for 6 h. After completion (monitored by TLC), the reaction mixture was diluted with water and extracted with EtOAc. The combined organic layers were dried over anhydrous Na₂SO₄, filtered, and concentrated under reduced pressure to give compound 1-5 (5.6 g, crude) as a brown solid, which was used in the next step without further purification. TLC: 50% EtOAc/heptane (R_{f}: 0.5). MS (ESI): calcd. for C₁₀H₁₄FIN₂O₂S: 371.98; Found: 373.06 [M+1]+. ¹H NMR (400 MHz, DMSO-d₆): δ 7.46-7.90 (m, 2H), 7.18 (d, J=6.63 Hz, 1H), 5.45 (s, 2H), 1.12 (s, 9H) ppm.

Step 5. Synthesis of tert-butyl (4-(2-amino-4-(N-(tert-butyl) sulfamoyl)-5-fluorophenyl) but-3-yn-1-yl) carbamate (1-6). To a stirred solution of compound 1-5 (2.6 g, 7.0 mmol) in THF (26 mL) were added tert-butyl but-3-yn-1-ylcarbamate (1.8 g, 10 mmol) and CuI (0.13 g, 0.70 mmol) followed by triethylamine (2.9 mL, 21 mmol) and the reaction mixture was purged under nitrogen atmosphere for 10 min. To this resulting solution was added Pd(PPh₃)₂Cl₂ (0.51 g, 0.70 mmol) under nitrogen atmosphere. The reaction mixture was stirred at 60° C. for 4 h. After completion (monitored by TLC), the reaction mixture was cooled to room temperature, diluted with water, and extracted with EtOAc. The combined organic layers were dried over anhydrous Na₂SO₄, filtered, and concentrated under reduced pressure. The crude product obtained was purified by CombiFlash column chromatography [eluting with 10% EtOAc in heptane] to give compound 1-6 (1.6 g, 55%) as a brown solid. TLC: 70% EtOAc/heptane (R_{f}: 0.2). MS (ESI): calcd. for C₁₉H₂₈FN₃O₄S: 413.18; Found: 414.28 [M+1]+. ¹H NMR (400 MHz, DMSO-d₆): δ 7.59 (s, 1H), 7.42-7.52 (m, 1H), 7.07-7.17 (m, 2H), 5.59 (s, 2H), 3.19 (q, J=6.36 Hz, 2H), 2.59 (t, J=6.60 Hz, 2H), 1.39 (s, 9H), 1.14 (s, 9H) ppm.

Step 6. Synthesis of tert-butyl (2-(6-(N-(tert-butyl) sulfamoyl)-5-fluoro-1H-indol-2-yl) ethyl) carbamate (1-7). In a sealed tube, compound 1-6 (3.2 g, 7.7 mmol) in DCE (32 mL) and copper(II) acetate (2.1 g, 12 mmol) were combined at room temperature. The reaction mixture was stirred at 150° C. for 3 h. After completion (monitored by TLC), the mixture was diluted with water and extracted with DCM. The combined organic layers were dried over anhydrous Na2SO4, filtered, and concentrated under reduced pressure. The crude compound was purified by CombiFlash column chromatography [eluting with 40-60% EtOAc in heptane] to give compound 1-7 (1.4 g, 44%) as an off-white solid. TLC: 80% EtOAc/heptane (R_{f}: 0.5). MS (ESI): calcd. for C₁₉H₂₈FN₃O₄S: 413.18; Found: 412.38 [M−1]⁻. ¹H NMR (400 MHz, DMSO-d₆): δ 11.43 (s, 1H), 7.70 (d, J=6.14 Hz, 1H), 7.39 (s, 1H), 7.34 (d, J=11.40 Hz, 1H), 6.96 (s, 1H), 6.28 (s, 1H), 3.24-3.28 (m, 2H), 2.87 (t, J=7.02 Hz, 2H), 1.36 (s, 9H), 1.08 (s, 9H) ppm.

Step 7. Synthesis of 2-(2-aminoethyl)-N-(tert-butyl)-5-fluoroindoline-6-sulfonamide (1-8). To a stirred solution of compound 1-7 (0.2 g, 0.484 mmol) in TFA (2 mL), borane-tetrahydrofuran complex (2.4 mL, 2.42 mmol, 1.0 mol/L in THF) was added at 0° C. The reaction mixture was stirred at room temperature for 4 h. After completion (monitored by TLC), the mixture was quenched with saturated NaHCO₃ solution, and extracted with EtOAc. The combined organic layers were washed with water, dried over anhydrous Na₂SO₄, filtered, and concentrated under reduced pressure to give compound 1-8 (70 mg, crude) as an off-white solid, which was used in the next step without purification. TLC: 10% MeOH/DCM (R_{f}: 0.1). MS (ESI): calcd. for C₁₄H₂₂FN₃O₂S: 315.14; Found: 316.29 [M+1]+. ¹H NMR (400 MHz, DMSO-d₆): δ 7.43 (s, 1H), 7.00 (d, J=10.0 Hz, 1H), 6.76 (d, J=5.8 Hz, 1H), 5.92 (s, 1H), 3.95-3.81 (m, 1H), 3.09 (dd, J=9.1, 16.6 Hz, 1H), 2.66-2.57 (m, 3H), 1.70-1.46 (m, 2H), 1.11 (s, 9H) ppm (two NH₂ protons are merged with DMSO).

Step 8. Synthesis of N-(tert-butyl)-7-fluoro-1-oxo-1,2,3,4,4a,5-hexahydropyrimido[1,6-a]indole-8-sulfonamide (1-9). To a stirred solution of compound 1-8 (70 mg, 0.222 mmol) in THF (2 mL) under nitrogen atmosphere, was added CDI (74 mg, 0.444 mmol). The reaction mixture was stirred at room temperature for 2 h. After completion (monitored by TLC), the reaction mixture was concentrated under reduced pressure. The residue was dissolved in Toluene (3 mL) and stirred at 100° C. for 2 h. Upon completion (monitored by TLC), the reaction mixture was diluted with water and extracted with DCM. The combined organic layers were washed with 10% citric acid and extracted with DCM, dried over anhydrous Na₂SO₄, filtered, and concentrated under reduced pressure to give compound 1-9 (60 mg, crude) as an off white solid, which was used in the next step without further purification. TLC: 5% MeOH/DCM (R_{f}: 0.5). MS (ESI): calcd. for C₁₅H₂₀FN₃O₃S: 341.12; Found: 342.28 [M+1]+. ¹H NMR (400 MHz, DMSO-d₆): δ 8.10 (d, J=6.58 Hz, 1H), 7.58 (s, 1H), 7.14-7.24 (m, 1H), 6.84 (s, 1H), 4.22-4.34 (m, 1H), 3.17-3.23 (m, 2H), 2.92 (dd, J=16.44, 10.74 Hz, 1H), 2.24-2.30 (m, 1H), 2.21-2.14 (m, 1H), 1.70-1.84 (m, 1H), 1.10 (s, 9H) ppm.

Step 9. Synthesis of N-(tert-butyl)-7-fluoro-1-oxo-2-(4-(pyridin-2-yl) phenyl)-1,2,3,4,4a,5-hexahydropyrimido[1,6-a]indole-8-sulfonamide (1-10). To a stirred solution of compound 1-9 (60 mg, 0.176 mmol) in 1,4-dioxane (5 mL) were added 2-(4-bromophenyl) pyridine (62 mg, 0.264 mmol) and Cs₂CO₃ (0.172 g, 0.527 mmol). The reaction mixture was purged with nitrogen gas for 10 min. To this resulting solution, Xantphos (21 mg, 0.035 mmol) and Pd₂(dba)₃ (17 mg, 0.018 mmol) were added under nitrogen atmosphere. The reaction mixture was stirred at 130° C. for 3 h in a microwave. After completion (monitored by TLC), the reaction mixture was cooled to room temperature, diluted with water, and extracted with EtOAc followed by brine. The combined organic layers were dried over anhydrous Na₂SO₄, filtered, and concentrated under reduced pressure. The crude compound was purified by CombiFlash column chromatography [eluting with 5% MeOH in DCM] to afford the title compound 1-10 (30 mg, 27%) as an off white solid. TLC: 5% MeOH/DCM (R_{f}: 0.5). MS (ESI): calcd. for C₂₆H₂₇FN₄O₃S: 494.18; Found: 495.84 [M+1]⁺.

Step 10. Synthesis of 7-fluoro-1-oxo-2-(4-(pyridin-2-yl) phenyl)-1,2,3,4,4a,5-hexahydropyrimido[1,6-a]indole-8-sulfonamide (Example 1). In a sealed tube, compound 1-10 (30 mg, 0.061 mmol) in 1,2-Dichloroethane (1 mL) and trifluoroacetic acid (1 mL) were combined at 0° C. under nitrogen atmosphere. The reaction mixture was slowly warmed to room temperature and allowed to stir at 60° C. for 4 h in. After completion (monitored by TLC), the reaction mixture was cooled to room temperature, saturated with NaHCO₃ solution, and extracted with EtOAc. The combined organic layers were washed with water dried over anhydrous Na₂SO₄, filtered, and concentrated under reduced pressure. The crude product obtained was purified by CombiFlash column chromatography [eluting with 5% MeOH in DCM] to give Example 1 (6 mg, 23%) as an off-white solid. TLC: 10% MeOH/DCM (R_{f}: 0.3).

### Examples 2 and 3. (S)-7-Fluoro-1-oxo-2-(4-(pyridin-2-yl)phenyl)-1,2,3,4,4a,5-hexahydropyrimido[1,6-a]indole-8-sulfonamide (Example 2) and (R)-7-fluoro-1-oxo-2-(4-(pyridin-2-yl)phenyl)-1,2,3,4,4a,5-hexahydropyrimido[1,6-a]indole-8-sulfonamide (Example 3)

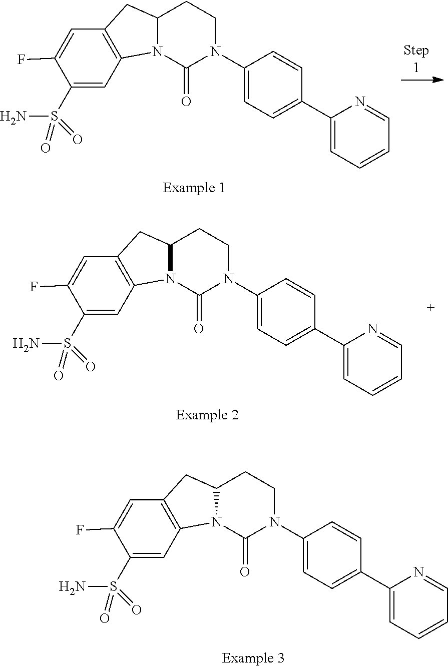

Example 1 (45 mg) was subjected to chiral preparative HPLC under the following conditions: Chiral Preparative SFC Method: Column IH (30×250 mm, 5u). Mobile phase A: 0.05% FA in hexane, Mobile phase B: DCM-MeOH (1:1). Eluent: (A:B, 75:35), Total Flow rate (42 mL/min). Fractions were collected and lyophilized to give Example 2 (4 mg) and Example 3 (4 mg) as off-white solids.

Table 1 shows structures and analytical data for representative exemplified compounds of the present invention. These compounds were prepared according to the synthetic schemes described above and using procedures known to those of ordinary skill in the art.

TABLE 1
Representative exemplified compounds of the present invention

| | | MS | |
|---|---|---|---|
| Example | Structure | [M + 1]⁺ | ¹H NMR |
| 1 | 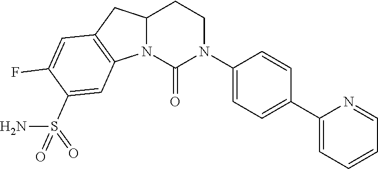 [SMILES: NS(=O)(=O)c1cc2c(cc1F)CC1CCN(c3ccc(-c4ccccn4)cc3)C(=O)N21] | 439.77 | ¹H NMR (400 MHz, DMSO-d₆): δ 8.67 (d, J = 3.91 Hz, 1H), 8.20 (d, J = 6.60 Hz, 1H), 8.09 (d, J = 8.68 Hz, 2H), 7.97 (d, J = 7.95 Hz, 1H), 7.85-7.92 (m, 1H), 7.50-7.56 (m, 4H), 7.28-7.37 (m, 2H), 4.54 (q, J = 11.53 Hz, 1H), 3.74-3.98 (m, 2H), 3.05 (dd, J = 16.51, 10.27 Hz, 2H), 1.96-2.23 (m, 2H) ppm |
| 2 | 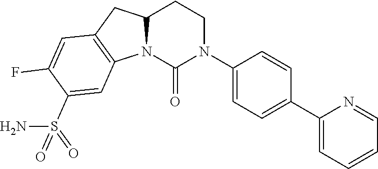 | 439.74 | ¹H NMR (400 MHz, DMSO-d₆): δ 8.67-8.65 (m, 1H), 8.21 (d, J = 6.63 Hz, 1H), 8.09 (d, J = 8.75 Hz, 2H), 7.97 (d, J = 8.4 Hz, 1H), 7.83-7.93 (m, 1H), 7.49-7.57 (m, 4H), 7.27-7.39 (m, 2H), 4.49-4.58 (m, 1H), 3.92-3.89 (m, 1H), 3.79- 3.75 (m, 1H), 3.33-3.39 (m, 1H), 3.08-3.01 (m, 1H), 2.42-2.38 (m, 1H), 2.08-2.24 (m, 1H) ppm |
| 3 | 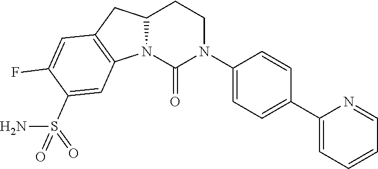 | 439.74 | ¹H NMR (400 MHz, DMSO-d₆): δ 8.67 (d, J = 4.00 Hz, 1H), 8.21 (d, J = 6.63 Hz, 1H), 8.09 (d, J = 8.63 Hz, 2H), 7.97 (d, J = 8.00 Hz, 1H), 7.84-7.93 (m, 1H), 7.48-7.57 (m, 4H), 7.26-7.40 (m, 2H), 4.49-4.59 (m, 1H), 3.95-3.88 (m, 1H), 3.70- 3.85 (m, 1H), 3.33-3.41 (m, 1H), 3.05 (dd, J = 16.57, 10.57 Hz, 1H), 2.41 (d, J = 11.88 Hz, 1H), 2.08- 2.24 (m, 1H) ppm |
| 4 | 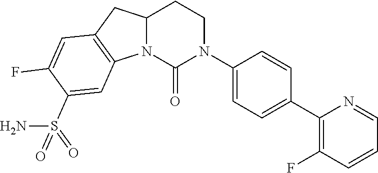 [SMILES: NS(=O)(=O)c1cc2c(cc1F)CC1CCN(c3ccc(-c4ncccc4F)cc3)C(=O)N21] | 457.71 | ¹H NMR (400 MHz, DMSO-d₆): δ 8.54-8.58 (m, 1H), 8.21 (d, J = 6.63 Hz, 1H), 7.94 (dd, J = 8.63, 1.50 Hz, 2H), 7.80-7.87 (m, 1H), 7.53-7.58 (m, 4H), 7.44-7.51 (m, 1H), 7.30 (d, J = 9.76 Hz, 1H), 4.54 (m, 1H), 3.93 (m, 1H), 3.79 (m, 1H), 3.33-3.41 (m, 1H), 3.05 (dd, J = 16.82, 10.69 Hz, 1H), 2.38-2.43 (m, 1H), 2.08-2.26 (m, 1H) ppm |
| 5 | 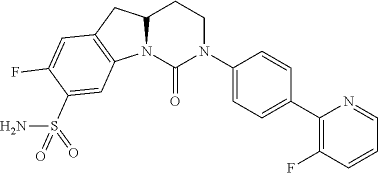 | 457.71 | ¹H NMR (400 MHz, DMSO-d₆): δ 8.53-8.58 (m, 1H), 8.21 (d, J = 6.63 Hz, 1H), 7.94 (dd, J = 8.63, 1.50 Hz, 2H), 7.79-7.89 (m, 1H), 7.52-7.58 (m, 4H), 7.44-7.51 (m, 1H), 7.30 (d, J = 9.76 Hz, 1H), 4.49-4.58 (m, 1H), 3.93-3.95 (m, 1H), 3.74-3.84 (m, 1H), 3.33-3.41 (m, 1H), 3.01-3.08 (m, 1H), 2.45- 2.40 (m, 1H), 2.07-2.23 (m, 1H) ppm |
| 6 | 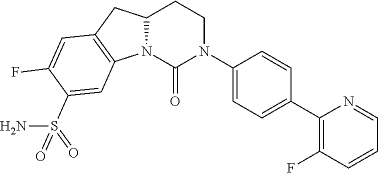 | 457.37 | ¹H NMR (400 MHz, DMSO-d₆): δ 8.56 (m, 1H), 8.21 (d, J = 6.63 Hz, 1H), 7.94 (dd, J = 8.63, 1.50 Hz, 2H), 7.78-7.89 (m, 1H), 7.46-7.60 (m, 5H), 7.30 (d, J = 9.76 Hz, 1H), 4.49-4.59 (m, 1H), 3.93 (m, 1H), 3.74-3.84 (m, 1H), 3.33-3.42 (m, 1H), 3.05 (dd, J = 16.57, 10.57 Hz, 1H), 2.36-2.46 (m, 1H), 2.07-2.26 (m, 1H) ppm |
| 7 | 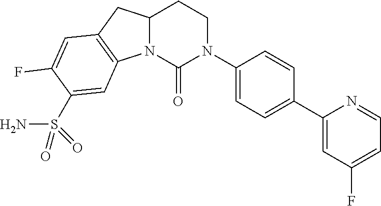 [SMILES: NS(=O)(=O)c1cc2c(cc1F)CC1CCN(c3ccc(-c4cc(F)ccn4)cc3)C(=O)N21] | 457.37 | ¹H NMR (400 MHz, DMSO-d₆): δ 8.69 (dd, J = 9.13, 5.63 Hz, 1H), 8.21 (d, J = 6.63 Hz, 1H), 8.14 (d, J = 8.75 Hz, 2H), 7.93 (dd, J = 11.13, 2.25 Hz, 1H), 7.51-7.57 (m, 4H), 7.25-7.33 (m, 2H), 4.49-4.58 (m, 1H), 3.93 (m, 1H), 3.78 (m, 1H), 3.33-3.41 (m, 1H), 3.05 (dd, J = 16.57, 10.57 Hz, 1H), 2.38- 2.42 (m, 1H), 2.06-2.23 (m, 1H) ppm |
| 8 | 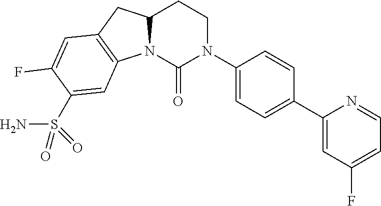 | 457.33 | ¹H NMR (400 MHz, DMSO-d₆): δ 8.69 (dd, J = 9.26, 5.63 Hz, 1H), 8.18-8.23 (m, 1H), 8.14 (d, J = 8.63 Hz, 2H), 7.93 (dd, J = 11.13, 2.25 Hz, 1H), 7.54 (d, J = 8.63 Hz, 4H), 7.24-7.34 (m, 2H), 4.48-4.58 (m, 1H), 3.86-3.98 (m, 1H), 3.78 (dd, J = 11.69, 3.31 Hz, 1H), 3.35- 3.39 (m, 1H), 3.09-3.01 (m, 1H), 2.39-2.42 (m, 1H), 2.07-2.32 (m, 1H) ppm |
| 9 | 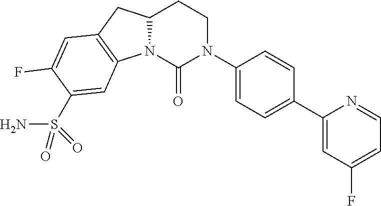 | 457.23 | ¹H NMR (400 MHz, DMSO-d₆): δ 8.69 (dd, J = 9.19, 5.69 Hz, 1H), 8.21 (d, J = 6.4 Hz, 1H), 8.14 (d, J = 8.76 Hz, 2H), 7.93 (dd, J = 11.13, 2.25 Hz, 1H), 7.51-7.57 (m, 4H), 7.23-7.35 (m, 2H), 4.49-4.58 (m, 1H), 3.89-3.96 (m, 1H), 3.78 (dd, J = 11.82, 3.19 Hz, 1H), 3.34- 3.39 (m, 1H), 3.00-3.10 (m, 1H), 2.38-2.43 (m, 1H), 2.10-2.21 (m, 1H) ppm |
| 10 | 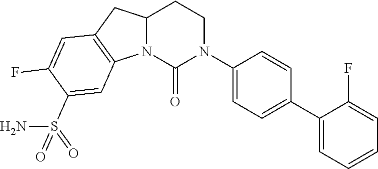 [SMILES: NS(=O)(=O)c1cc2c(cc1F)CC1CCN(c3ccc(-c4ccccc4F)cc3)C(=O)N21] | 456.25 | ¹H NMR (400 MHz, DMSO-d₆): δ 8.20 (d, J = 6.63 Hz, 1H), 7.49- 7.60 (m, 7H), 7.39-7.46 (m, 1H), 7.28-7.35 (m, 3H), 4.49-4.59 (m, 1H), 3.84-3.98 (m, 1H), 3.89-3.87 (m, 1H), 3.37-3.35 (m, 1H), 3.08- 3.01 (m, 1H), 2.41-2.45 (m, 1H), 2.10-2.23 (m, 1H) ppm |
| 11 | 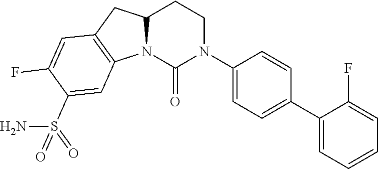 | 456.25 | ¹H NMR (400 MHz, DMSO-d₆): δ 8.20 (d, J = 6.63 Hz, 1H), 7.44- 7.61 (m, 8H), 7.26-7.37 (m, 3H), 4.49-4.59 (m, 1H), 3.91 (m, 1H), 3.78-3.74 (m, 1H), 3.35-3.41 (m, 1H), 3.05 (m, 1H), 2.43-2.39 (m, 1H), 2.16 (m, 1H) ppm |
| 12 | 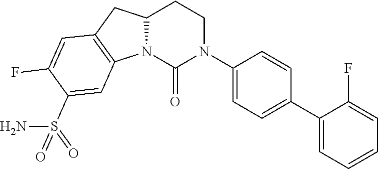 | 456.28 | ¹H NMR (400 MHz, DMSO-d₆): δ 8.20 (d, J = 6.63 Hz, 1H), 7.39- 7.59 (m, 8H), 7.28-7.35 (m, 3H), 4.49-4.59 (m, 1H), 3.91 (m, 1H), 3.76 (m, 1H), 3.35-3.41 (m, 1H), 3.05 (m, 1H), 2.41 (m, 1H), 2.16 (m, 1H) ppm |
| 13 | 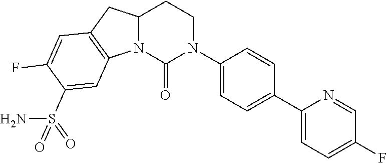 [SMILES: NS(=O)(=O)c1cc2c(cc1F)CC1CCN(c3ccc(-c4ccc(F)cn4)cc3)C(=O)N21] | 457.33 | ¹H NMR (400 MHz, DMSO-d₆): δ 8.66 (d, J = 3.00 Hz, 1H), 8.21 (d, J = 6.50 Hz, 1H), 8.03-8.08 (m, 3H), 7.80-7.86 (m, 1H), 7.54 (d, J = 3.50 Hz, 2H), 7.52 (s, 2H), 7.31 (d, J = 10.01 Hz, 1H), 4.48-4.60 (m, 1H), 3.86-3.96 (m, 1H), 3.75- 3.81 (m, 1H), 3.37 (d, J = 8.50 Hz, 1H), 3.05 (dd, J = 16.51, 11.01 Hz, 1H), 2.42 (d, J = 11.51 Hz, 1H), 2.12-2.22 (m, 1H) ppm |
| 14 | 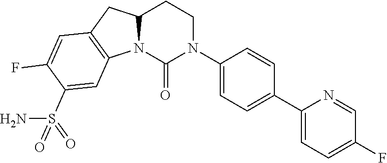 | | ¹H NMR (400 MHz, DMSO-d₆): δ 8.66 (d, J = 2.75 Hz, 1H), 8.21 (d, J = 6.50 Hz, 1H), 7.97-8.10 (m, 3H), 7.77-7.88 (m, 1H), 7.54 (d, J = 4.00 Hz, 2H), 7.51 (s, 2H), 7.30 (d, J = 9.76 Hz, 1H), 4.54 (J = 10.76 Hz, 1H), 3.87-3.98 (m, 1H), 3.77 (dd, J = 11.76, 3.25 Hz, 1H), 3.37 (d, J = 8.50 Hz, 1H), 3.05 (dd, J = 16.76, 10.76 Hz, 1H), 2.42 (d, |
| | | | J = 12.26 Hz, 1H), 2.08-2.25 (m, |
| | | | 1H) ppm |
| 15 | 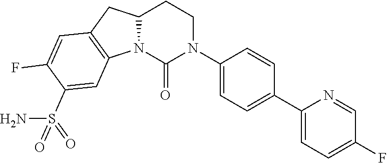 | 457.33 | ¹H NMR (400 MHz, DMSO-d₆): δ 8.66 (d, J = 2.75 Hz, 1H), 8.21 (d, J = 6.50 Hz, 1H), 8.06 (d, J = 8.76 Hz, 3H), 7.79-7.86 (m, 1H), 7.54 (d, J = 4.00 Hz, 2H), 7.51 (s, 2H), 7.30 (d, J = 9.76 Hz, 1H), 4.54 (d, J = 10.51 Hz, 1H), 3.84-3.97 (m, 1H), 3.77 (dd, J = 11.51, 3.50 Hz, 1 H), 3.37 (d, J = 8.76 Hz, 1H), 3.05 (dd, J = 16.63, 10.63 Hz, 1H), |
| | | | 2.42 (d, J = 12.26 Hz, 1H), 2.10- |
| | | | 2.22 (m, 1H) ppm |
| 16 | 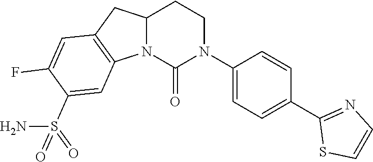 [SMILES: NS(=O)(=O)c1cc2c(cc1F)CC1CCN(c3ccc(-c4nccs4)cc3)C(=O)N21] | 445.27 | ¹H NMR (400 MHz, DMSO-d₆): δ 8.17 (d, J = 6.50 Hz, 1H), 7.87- 7.99 (m, 3H), 7.74 (d, J = 3.00 Hz, 1H), 7.52 (s, 2H), 7.50 (m, 2H), 7.26 (d, J = 9.51 Hz, 1H), 4.40- 4.57 (m, 1H), 3.82-3.94 (m, 1H), 3.72-3.78 (m, 1H), 3.32 (d, J = 8.50 Hz, 1H), 3.01 (dd, J = 16.76, 10.26 Hz, 1H), 2.35-2.41 (m, 1H), 2.06-2.18 (m, 1H) ppm |
| 17 | 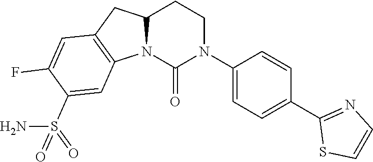 | 445.23 | ¹H NMR (400 MHz, DMSO-d₆): δ 8.18 (d, J = 6.58 Hz, 1H), 7.89- 7.96 (m, 2H), 7.76 (d, J = 3.07 Hz, 1H), 7.46-7.56 (m, 3H), 7.28 (d, J = 9.65 Hz, 1H), 4.37-4.66 (m, 1H), 3.86-3.94 (m, 1H), 3.73-3.79 (m, 1H), 3.02 (dd, J = 16.66, 10.52 Hz, 1H), 2.37-2.39 (m, 1H) 2.00- 2.20 (m, 2H) ppm (Sulfonamide NH₂ not observed due to moisture) |
| 18 | 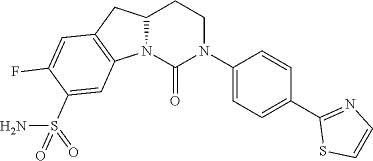 | 445.23 | ¹H NMR (400 MHz, DMSO-d₆): δ 8.18 (d, J = 6.58 Hz, 1H), 7.90- 7.94 (m, 2H), 7.76 (d, J = 3.07 Hz, 1H), 7.48-7.59 (m, 3H), 7.28 (d, J = 9.65 Hz, 1H), 4.42-4.58 (m, 1H), 3.83-3.96 (m, 1H), 3.76 (d, J = 8.77 Hz, 1H), 3.02 (dd, J = 16.01, 10.74 Hz, 1H), 2.37-2.39 (m, 1H), 2.06-2.19 (m, 2H) ppm (Sulfonamide NH₂ not observed possibly due to moisture) |
| 19 | 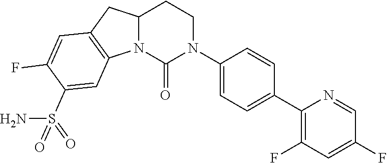 [SMILES: NS(=O)(=O)c1cc2c(cc1F)CC1CCN(c3ccc(-c4ncc(F)cc4F)cc3)C(=O)N21] | 475.27 | ¹H NMR (400 MHz, DMSO-d₆): δ 8.64 (d, J = 2.25 Hz, 1H,) 8.21 (d, J = 6.50 Hz, 1H), 8.03-8.11 (m, 1H), 7.88 (dd, J = 8.51, 1.50 Hz, 2H), 7.55-7.58 (m, 2H), 7.54 (s, 2H), 7.30 (d, J = 9.76 Hz, 1H), 4.48-4.61 (m, 1H), 3.86-3.96 (m, 1H), 3.75-3.82 (m, 1H), 3.35-3.40 (m, 1H), 3.05 (dd, J = 16.63, 10.63 Hz, 1H), 2.40-2.45 (m, 1H), 2.10- 2.22 (m, 1H) ppm |
| 20 | 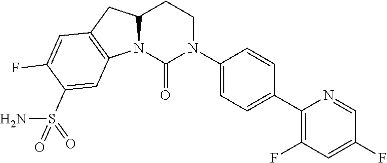 | 475.23 | ¹H NMR (400 MHz, DMSO-d₆): δ 8.65 (d, J = 2.25 Hz, 1H), 8.21 (d, J = 6.50 Hz, 1H), 8.01-8.11 (m, 1H), 7.81-7.91 (m, 2H), 7.55-7.58 (m, 2H), 7.54 (s, 2H), 7.30 (d, J = 9.76 Hz, 1H), 4.49-4.59 (m, 1H), 3.88-3.98 (m, 1H), 3.75-3.83 (m, 1H), 3.31-3.36 (m, 1H), 3.05 (dd, J = 16.88, 10.63 Hz, 1H), 2.43- 2.46 (m, 1H), 2.11-2.22 (m, 1H) ppm |
| 21 | 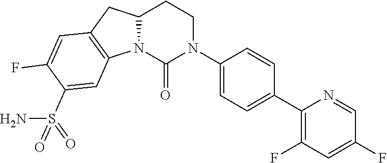 | 475.23 | ¹H NMR (400 MHz, DMSO-d₆): δ 8.64 (d, J = 2.00 Hz, 1H), 8.21 (d, J = 6.50 Hz, 1H), 8.04-8.11 (m, 1H), 7.88 (d, J = 7.00 Hz, 2H), 7.54-7.59 (m, 2H), 7.53 (s, 2H), 7.30 (d, J = 9.76 Hz, 1H), 4.47- 4.61 (m, 1H), 3.88-3.96 (m, 1H), 3.74-3.82 (m, 1H), 3.34-3.40 (m, 1H), 3.05 (dd, J = 16.01, 11.01 Hz, 1H), 2.39-2.45 (m, 1H), 2.08-2.23 (m, 1H) ppm |
| 22 | 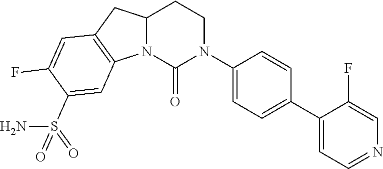 [SMILES: NS(=O)(=O)c1cc2c(cc1F)CC1CCN(c3ccc(-c4ccncc4F)cc3)C(=O)N21] | 451.71 | ¹H NMR (400 MHz, DMSO-d₆): δ 8.66 (s, 1H), 8.51 (d, J = 4.56 Hz, 1H), 8.20 (d, J = 6.63 Hz, 1H), 7.64-7.73 (m, 3H), 7.58 (d, J = 8.29 Hz, 2H), 7.55 (s, 2H), 7.31 (d, J = 9.95 Hz, 1H), 4.48-4.62 (m, 1H), 3.86-4.01 (m, 1H), 3.75-3.82 (m, 1H), 3.35-3.38 (m, 1H), 3.05 (dd, J = 16.17, 10.78 Hz, 1H), 2.40-2.45 (m, 1H), 2.11-2.21 (m, 1H) ppm |
| 23 | 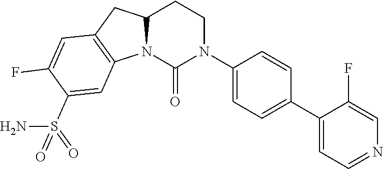 | 457.71 | ¹H NMR (400 MHz, DMSO-d₆): δ 8.66 (d, J = 2.75 Hz, 1H), 8.50- 8.52 (m, 1H), 8.20 (d, J = −6.50 Hz, 1H), 7.64-7.73 (m, 3H), 7.58 (d, J = 8.75 Hz, 2H), 7.54 (s, 2H), 7.30 (d, J =9.76 Hz, 1H), 4.49-4.60 (m, 1H), 3.88-4.00 (m, 1H), 3.74- 3.82 (m, 1H), 3.32-3.38 (m, 1H), 3.05 (dd, J = 16.63, 10.63 Hz, 1H), 2.39-2.46 (m, 1H), 2.12-2.23 (m, 1H) ppm |
| 24 | 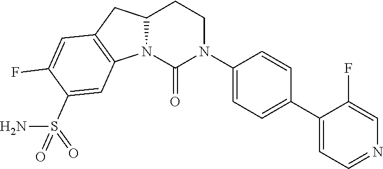 | 457.71 | ¹H NMR (400 MHz, DMSO-d₆): δ 8.66 (d, J = 2.75 Hz, 1H), 8.50- 8.53 (m, 1H), 8.20 (d, J = 6.50 Hz, 1H), 7.64-7.72 (m, 3H,) 7.58 (d, J = 8.75 Hz, 2H), 7.54 (s, 2H), 7.31 (d, J = 9.76 Hz, 1H), 4.47-4.59 (m, 1H), 3.89-3.97 (m, 1H), 3.75-3.83 (m, 1H), 3.33-3.39 (m, 1H), 3.05 (dd, J = 16.76, 10.51 Hz, 1H), 2.39-2.46 (m, 1H), 2.12-2.21 (m, 1H) ppm |
| 25 | 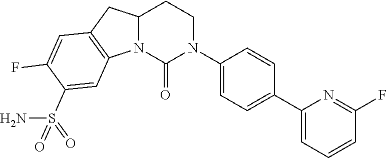 [SMILES: NS(=O)(=O)c1cc2c(cc1F)CC1CCN(c3ccc(-c4cccc(F)n4)cc3)C(=O)N21] | 457.20 | ¹H NMR (400 MHz, DMSO-d₆): δ 7.84-8.32 (m, 5H), 7.55-7.58 (m, 2H), 7.54 (s, 2H), 7.30 (d, J = 9.12 Hz, 1H), 7.12 (d, J = 6.22 Hz, 1H), 4.38-4.68 (m, 1H), 3.85-4.00 (m, 1H), 3.72-3.82 (m, 1H), 2.98-3.14 (m, 1H), 2.37-2.41 (m, 2H), 2.07- 2.21 (m, 1H) ppm |
| 26 | 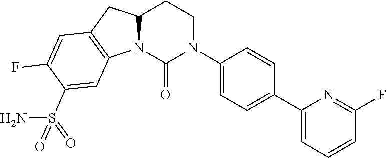 | 457.20 | ¹H NMR (400 MHz, DMSO-d₆): δ 8.21 (d, J = 6.50 Hz, 1H), 8.04- 8.13 (m, 3H), 7.91-7.98 (m, 1H), 7.55-7.57 (m, 2H), 7.54 (s, 2H), 7.31 (d, J = 9.51 Hz, 1H), 7.07- 7.17 (m, 1H), 4.45-4.62 (m, 1H), 3.88-3.99 (m, 1H), 3.73-3.84 (m, 1H), 3.36-3.43 (m, 1H), 3.05 (dd, J = 16.38, 10.63 Hz, 1H), 2.38- 2.48 (m, 1H), 2.08-2.24 (m, 1H) ppm |
| 27 | 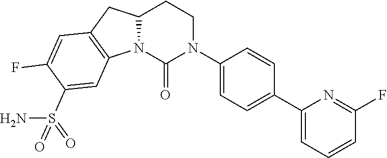 | 457.23 | ¹H NMR (400 MHz, DMSO-d₆): δ 8.21 (d, J = 6.50 Hz, 1H), 8.03- 8.09 (m, 3H), 7.94 (dd, J = 7.50, 2.50 Hz, 1H), 7.54-7.56 (m, 2H) 7.54 (s, 2H), 7.30 (d, J = 10.01 Hz, 1H), 7.12 (dd, J = 8.00, 2.50 Hz, 1H), 4.47-4.59 (m, 1H), 3.86-3.96 (m, 1H), 3.74-3.82 (m, 1H), 3.05 (dd, J = 17.01, 10.51 Hz, 1H), 2.37-2.46 (m, 1H), 2.11-2.22 (m, |
| | | | 1H) ppm (one CH proton is |
| | | | merged with solvent peak) |

### Biological Assay Data

### Cell Culture

Vero cells were cultured in Dulbecco's Modified Eagle Medium (DMEM) supplemented with 10% fetal bovine serum and 100 units/mL penicillin and streptomycin. The cells were passaged 2-3 times per week to maintain sub-confluent densities.

### Assays

### HSV-1 Antiviral Assay

Vero cells were seeded into 96-well plates at a density of 2.5×10³ cells per well and allowed to attach overnight. Following attachment, the media was replaced with 50 uL of infection medium (DMEM supplemented with 2% fetal bovine serum and 100 units/mL penicillin and streptomycin). A Tecan D300e digital dispenser was then used to add compounds to the culture using an 8-point 3-fold serial dilution format. The DMSO concentration was normalized to 0.5% for all treatments. Following compound addition, 50 μL of infection medium containing 80 TCID₅₀ HSV-1 was added to the cells and incubated at 37° C. for 4 days. After the incubation, the plates were equilibrated to room temperature, the media was removed, and 60 of a 1:1 dilution of Cell titer glow and phosphate buffered saline was added to the cells. Following a 5-minute incubation, cell viability was quantified by measuring luminance using a Tecan Infinite M1000 Pro plate reader.

### HSV-2 Antiviral Assay

Vero cells were seeded into 96-well plates at a density of 1.0×10⁴ cells per well and allowed to attach overnight. Following attachment, the media was replaced with 50 μL of infection medium (DMEM supplemented with 2% fetal bovine serum and 100 units/mL penicillin and streptomycin). A Tecan D300e digital dispenser was then used to add compounds to the culture using an 8-point 3-fold serial dilution format. The DMSO concentration was normalized to 0.5% for all treatments. Following compound addition, 50 μL of infection medium containing 160 TCID₅₀ HSV-2 G strain was added to the cells and incubated at 37° C. for 5 days. After the incubation, 10 μL/well of WST-8 chromogenic reagent was added and the plates incubated at 37° C. for 3 hours. Following the incubation, cell viability was quantified by measuring the absorbance at 460 nm and 620 nm using a Tecan Infinite M1000 Pro plate reader.

Table 2 provides assay data for exemplified compounds of the invention grouped in the following ranges: A indicates EC₅₀<100 nM; B indicates 100≤EC₅₀<1,000 nM; NA indicates data not available.

TABLE 2
Assay data for exemplified compounds of the invention.

| Example | HSV-1 | HSV-2 |
|---|---|---|
| 1 | A | A |
| 2 | A | A |
| 3 | NA | A |
| 4 | NA | A |
| 5 | A | A |
| 6 | NA | A |
| 7 | NA | A |
| 8 | A | A |
| 9 | NA | A |
| 10 | A | A |
| 11 | A | A |
| 12 | A | A |
| 13 | B | B |
| 14 | NA | B |
| 15 | NA | A |
| 16 | NA | A |
| 17 | NA | A |
| 18 | NA | A |
| 19 | NA | B |
| 20 | NA | B |
| 21 | NA | B |
| 22 | NA | B |
| 23 | NA | B |
| 24 | NA | B |
| 25 | NA | A |
| 26 | NA | A |
| 27 | NA | A |

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A compound of Formula I 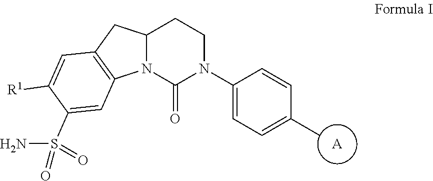
or a pharmaceutically acceptable salt thereof, wherein: 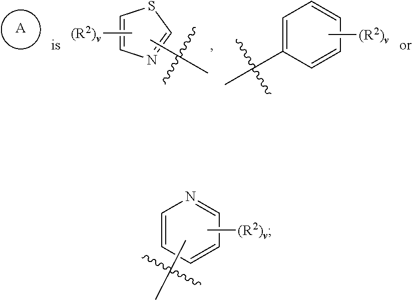
R¹ is hydrogen, CN, halo or methyl;
R² is independently selected for each occurrence from the group consisting of halo, CN, C₁₋₄alkyl and haloC₁₋₄alkyl; and
vis, 1 or 2;

2. The compound of claim 1, or a pharmaceutically acceptable salt thereof, wherein

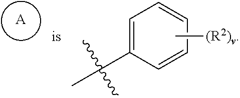

3. The compound of claim 1, or a pharmaceutically acceptable salt thereof, wherein

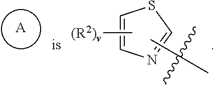

4. The compound of claim 1, or a pharmaceutically acceptable salt thereof, wherein

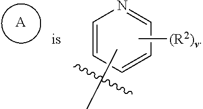

5. The compound of claim 4, or a pharmaceutically acceptable salt thereof, wherein

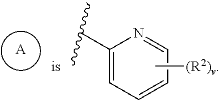

6. The compound according to claim 1, or a pharmaceutically acceptable salt thereof, wherein v is 1 or 2 and R² is F for each occurrence.

7. The compound according to claim 1, or a pharmaceutically acceptable salt thereof, wherein R¹ is F.

8. A compound selected from:

| Example | Structure |
|---|---|
| 1 | 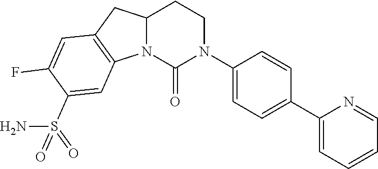 [SMILES: NS(=O)(=O)c1cc2c(cc1F)CC1CCN(c3ccc(-c4ccccn4)cc3)C(=O)N21] |
| 2 | 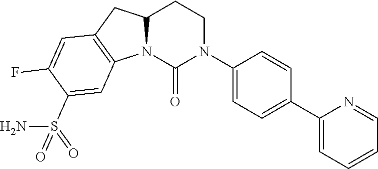 |
| 3 | 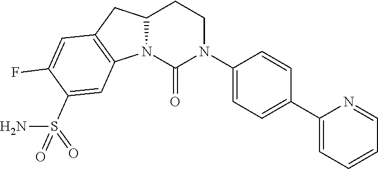 |
| 4 | 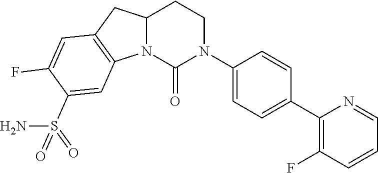 [SMILES: NS(=O)(=O)c1cc2c(cc1F)CC1CCN(c3ccc(-c4ncccc4F)cc3)C(=O)N21] |
| 5 | 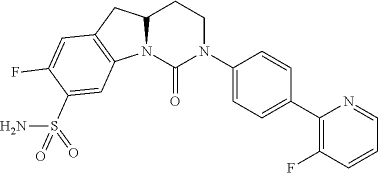 |
| 6 | 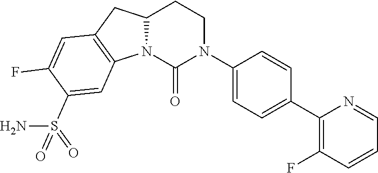 |
| 7 | 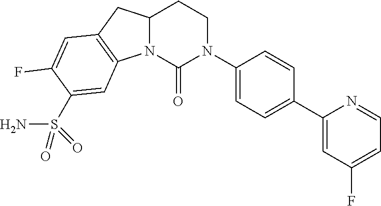 [SMILES: NS(=O)(=O)c1cc2c(cc1F)CC1CCN(c3ccc(-c4cc(F)ccn4)cc3)C(=O)N21] |
| 8 | 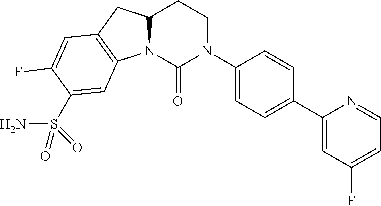 |
| 9 | 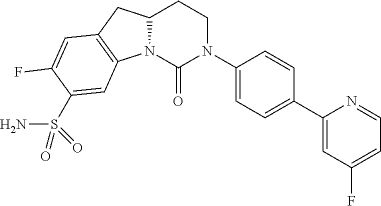 |
| 10 | 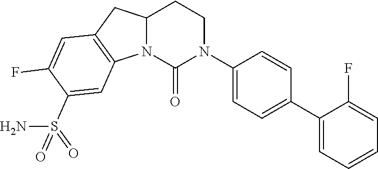 [SMILES: NS(=O)(=O)c1cc2c(cc1F)CC1CCN(c3ccc(-c4ccccc4F)cc3)C(=O)N21] |
| 11 | 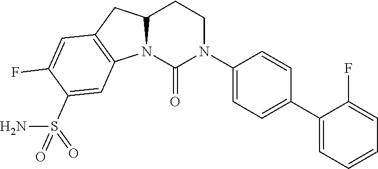 |
| 12 | 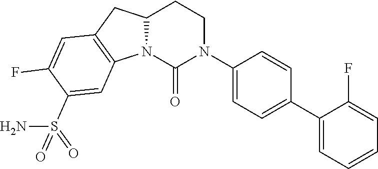 |
| 13 | 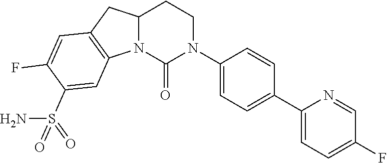 [SMILES: NS(=O)(=O)c1cc2c(cc1F)CC1CCN(c3ccc(-c4ccc(F)cn4)cc3)C(=O)N21] |
| 14 | 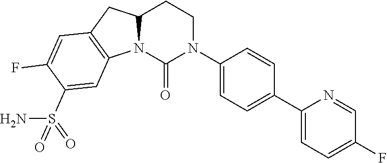 |
| 15 | 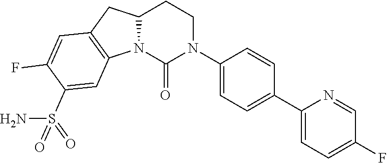 |
| 16 | 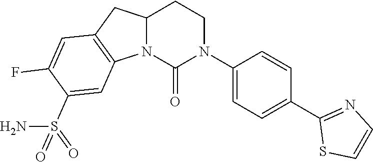 [SMILES: NS(=O)(=O)c1cc2c(cc1F)CC1CCN(c3ccc(-c4nccs4)cc3)C(=O)N21] |
| 17 | 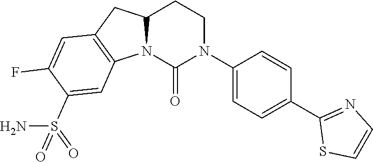 |
| 18 | 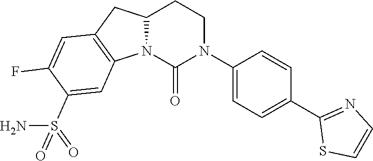 |
| 19 | 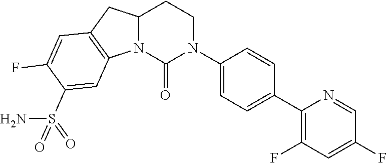 [SMILES: NS(=O)(=O)c1cc2c(cc1F)CC1CCN(c3ccc(-c4ncc(F)cc4F)cc3)C(=O)N21] |
| 20 | 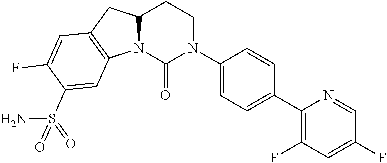 |
| 21 | 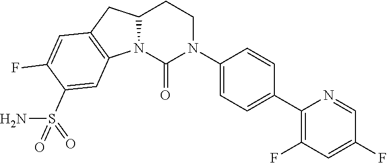 |
| 22 | 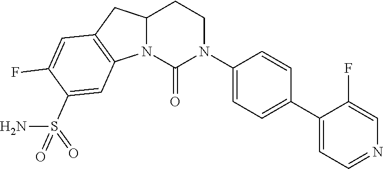 [SMILES: NS(=O)(=O)c1cc2c(cc1F)CC1CCN(c3ccc(-c4ccncc4F)cc3)C(=O)N21] |
| 23 | 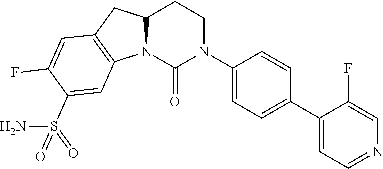 |
| 24 | 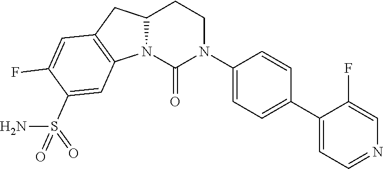 |
| 25 | 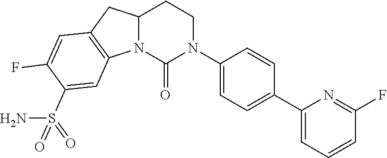 [SMILES: NS(=O)(=O)c1cc2c(cc1F)CC1CCN(c3ccc(-c4cccc(F)n4)cc3)C(=O)N21] |
| 26 | 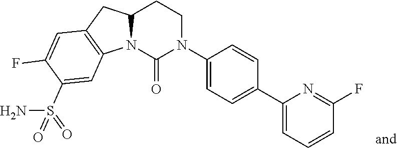 |
| 27 | 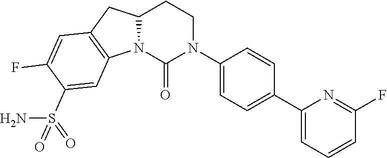 |

or a pharmaceutically acceptable salt thereof.

9. A pharmaceutical composition comprising a compound according to claim 1, or a pharmaceutically acceptable salt thereof, and a pharmaceutically acceptable excipient.

10. A method for the treatment or prophylaxis of an HSV infection in a subject in need thereof, the method comprising: administering to the subject a therapeutically effective amount of a compound according to claim 1, or a pharmaceutically acceptable salt thereof.

11. A method for the treatment or prophylaxis of an HSV infection in a subject in need thereof, the method comprising: administering to the subject a therapeutically effective amount of a pharmaceutical composition of claim 9.

12. The method of claim 10, wherein infection is an HSV-1 infection.

13. The method of claim 10, wherein infection is an HSV-2 infection.

14. A pharmaceutical composition comprising a compound according to claim 8, or a pharmaceutically acceptable salt thereof, and a pharmaceutically acceptable excipient.

15. A method for the treatment or prophylaxis of an HSV infection in a subject in need thereof, the method comprising: administering to the subject a therapeutically effective amount of a compound according to claim 8, or a pharmaceutically acceptable salt thereof.

16. A method for the treatment or prophylaxis of an HSV infection in a subject in need thereof, the method comprising: administering to the subject a therapeutically effective amount of a pharmaceutical composition of claim 14.

17. The method of claim 15, wherein infection is an HSV-1 infection.

18. The method of claim 15, wherein infection is an HSV-2 infection.
